(19) United States
(12) Patent Application Publication
(10) Pub. No.: US 2009/0048499 A1
(43) Pub. Date: Feb. 19, 2009

(54) Sensor film for transcutaneous insertion and a method for making the sensor film

(75) Inventor: Kristian Glejbol, Glostrup (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/665,427

(22) PCT Filed: Oct. 12, 2005
(86) PCT No.: PCT/EP2005/055175

§ 371 Date: May 19, 2008

Related U.S. Application Data
(60) Provisional application No. 60/624,272, filed on Nov. 2, 2004.

(30) Foreign Application Priority Data
Oct. 18, 2004 (DK) PA 2004 01591

(51) Int. Cl. A61B 5/00

(52) U.S. Cl. 600/309

(57) ABSTRACT

There is provided a thin film (1,2 or 1,3, FIGS. 1 and 2) for transcutaneous insertion by means of a needle assembly or the like. The sensor film comprises an elongate electrode part (1) which is integral with a contact part (2, 3). According to the invention the contact part (2, 3) extends to both sides of at least one axial plane that contains a longitudinal axis of the electrode part (1). Thereby it is obtained that a needle assembly containing the sensor film can be a compact unit while simultaneously the contact part is configured such that the opportunities of contacting and fixating and using the sensor film to advantage are improved considerably compared to the achievement of the prior art.

### FIELD OF THE INVENTION

This invention relates to the production of electrode assemblies suitable for use in electrochemical sensors, in particular for transcutaneous electrochemical sensors suitable for in vivo measurement of metabolites.

### BACKGROUND OF THE INVENTION

In recent years, a variety of electrochemical sensors have been developed for vivo measurements of metabolites. Most prominent among these glucose sensors have been developed for use for obtaining an indication of blood glucose (BG) levels in a diabetic patient.

BG information is of the utmost importance to diabetics, as these readings are instrumental for the adjustment of the treatment regimen. The conventional way to obtain BG information is applying minute amounts of blood to test strips. A new development is transcutaneous sensors where the sensor is implanted under the skin. As the sensor is in contact with biological fluids for a prolonged period of time the possibility of continuous measurements is opened. Continuous BG readings obtained with little or no delay is particularly useful in numerous ways. First of all the continuous monitoring will help to prevent hypoglycaemic incidents and thus contribute to a vast increase for the quality of life of the diabetic patient. Furthermore continuous BG readings may be used eg in conjunction with semi-automated medication infusion pumps of the external type or automated implantable medication infusion pumps, as generally described in U.S. Pat. Nos. 3,837,339, 4,245,634 and 4,515,584. This will allow the patient to have a near-normal lifestyle, thus eliminating or greatly minimizing the problems usually associated with diabetes.

Sensors utilised for measurement of blood glucose (BG) or other metabolites can be made in a number of different ways. In its simplest form the sensor is made with two separate electrodes placed near each other. The two electrodes designated working electrode (WE) and reference electrode (RE) serve different respective purposes. A sensor with more electrodes is designated “electrode assembly”.

It is common to most electrode assemblies that each electrode is electrically connected to a contact pad on a contact part of the sensor assembly through an elongated electrical conducting member referred to as a conductor track. Electrical contact is preferred at the two ends of each conductor track/each electrical connection. The conductor tracks are most often covered with a layer of insulating material (dielectric). At one end of the conductor track an area remains naked such that contact can be established to the supporting electrical circuits; such an end is in the following designated CPE (Contact Pad for Electronics). At the other end, an area is also left naked and serves as the electrode surface; this end is in the following designated electrode part of the sensor.

Various strategies exist for the production of electrode assemblies, e.g. as described in Urban and Jobst, in D. M. Fraser (Ed), Biosensors in the body, John Wiley & Sons, Chichester, UK, 1997, p. 197-216. One commonly used strategy is to dispose electrical conducting tracks or wires on flexible foils made by a dielectric material. Several methods exist for deposition of conducting tracks, including printing, etching of conducting layers covering the flexible foils or by direct vacuum plating of conducting structures. This is an expensive process, and when producing sensors it is important that the cost is reduced as much as possible without compromising the quality of the product. Although important to the production yield, this aspect is only seldom addressed in the literature. Owing to the low quantity of materials used for electrode assemblies, the production cost of these is roughly proportional to the area they take up during production. Thus, it is commercially important to reduce the used area/electrode assembly as much as possible.

As a consequence of the development within the field of electronics, today the patient is offered sophisticated and reliable signal processing. However, in some embodiments this may involve that the contact part must be larger that known so far in order to accommodate passive and/or active electric circuits. At the same time the comparatively costly sensor material must be used to advantage without waste.

As mentioned above, the most commonly used sensors are in the form of a thin film that can, in practice, be inserted subcutaneously by means of a needle or other rigid insertion means.

This technique is known ia from U.S. Pat. No. 5,390,671, where it is possible to withdraw the needle following insertion of the sensor due to the flexible sensor having a proximal segment and a distal segment, said proximate and distal segments being misaligned with respect to each other. An almost corresponding solution is found in U.S. Pat. No. 6,134,461, wherein the proximate region extends in a single lateral direction from the longitudinal axis of the distal region.

It is a common feature of these two prior art techniques that the area of the sensor which, during insertion of the needle, extends outside same is relatively small and leaves only few practically viable opportunities for creating contact between coupling terminals and the contact areas that are provided on the contact portion of the sensor. In the following, that area of the sensor will be designated the contact part, while the area which is situated inside the needle during insertion will be designated the electrode part.

It is the object of the invention to provide a flexible sensor, wherein the contact part is configured such that the opportunities of contacting and fixating and using the sensor film to advantage are improved considerably compared to the achievements of the prior art, while simultaneously the sensor assembly has to be a compact unit.

This object is accomplished in that the contact part extends to both sides of at least one axial plane that contains a longitudinal axis of the electrode part.

The contact part extending in this manner to both sides of the proximal extension of the electrode enhances the opportunities with regard to shaping of the contact part in such an manner—while maintaining a compact sensor—as to allow it to be more easily provided with passive or active circuits or be more easily laminated with other films and/or be more easily folded and shaped in such a manner that the contact part i.a. is able to stay clear of the proximal extension of the electrode part.

According to a preferred embodiment the contact area is Ω-shaped and, in principle, it is possible to include folding areas at several points along the contour of such contact area.

The sensor film being flexible enables the contact part to flex elastically in a direction away from the proximal extension of the electrode part, whereby the insertion needle can be mounted and withdrawn; however, the contact part may also be folded with ensuing permanent deformation, where the folding areas may have generatrices that may, on the one hand, be essentially in parallel with the electrode part and, on the other, be transversal to the electrode part. In this context, those points on the contact part where the points of contact are located may be in a plane essentially in parallel with the electrode part or essentially perpendicular or transversal to the electrode part, depending on what is most expedient for the relevant use. For instance, the points of contact may advantageously slide towards respective terminals during insertion to ensure good connection; however, there may also be cases where the contact pads are advantageously located on an area of the contact part which is perpendicular to the direction of insertion of the electrode part, viz in case it is desired, by the contacting, to penetrate an insulating layer in the points of contact.

On this comparatively large contact part, electric circuits can be arranged. On that comparatively large contact face, it is also an option to arrange active electronic circuits, and the fact that they are arranged at an area of the contact part which is essentially perpendicular to the electrode part enables sterilisation by means of electron irradiation essentially at right angles to the electrode part, whereby the direction of electrons is in parallel with the P-N transition faces in the active circuits, thereby avoiding that the electron irradiation causes damage thereto.

Usually the electrodes on the electrode part and the contact pads on the contact part are connected to each other by means of electric conductors, and according to one embodiment the conductors are exposed in line with the proximal extension of the electrode part, thereby enabling short-circuiting of the conductors by means of the needle, in particular during the sterilisation by means of electron beams. Thereby the build-up of static electricity is prevented which may otherwise destroy the active circuits.

The relatively large contact part also improves the options of laminating the sensors in more layers, and there will also be space for configuring one or more guide openings in the contact part for use in connection with automatic mounting. The relatively large area of the contact part can also be used to advantage for configuring guide openings in the film.

In brief, the comparatively large contact part situated to both sides of the electrode part provides many advantages compared to the prior art. According to a preferred embodiment the electrode part of the sensor film is twisted somewhere between the distal and the proximal ends, thereby causing these ends to extend in essentially mutually perpendicular planes, whereby the distal end of the electrode part is locked in the needle, whereby tissue or hair are unable to dislocate the sensor relative to the needle during insertion.

The invention also relates to a method of manufacturing a flexible sensor film having a distal end and a proximal end that are coherent at a contact part of the sensor film, which method is characterised in that sensor elements are provided on a carrier film, wherein each sensor element comprises a contact part having a recess that extends into the contact part and comprises an electrode part that extends in a direction away from the contact part in line with the recess; and wherein the sensor elements are situated in pairs opposite each other, whereby an electrode part from the one element in the pair of elements extends into the recess in the second element in the pair of elements.

In this manner very efficient utilisation of the expensive sensor film is accomplished and, essentially, it will be the need for contact/circuit area that determines the consumption of sensor film.

The area of the recess can be made comparatively large or comparatively small relative to the area of the contact area; it being understood, however, that a relatively large recess provides a relatively small, but rather flexible contact part, whereas a relatively small recess leaves a rather rigid and larger contact part. In connection with the manufacture of the sensors, eg application of glucose oxidase, the electrode parts are advantageously processed by being run linearly through a processing apparatus. Arrangement of the sensor elements on an elongate film, eg a roll supply of film, processing apparatuses may perform a continuous manufacture of sensor films.

When the sensors are manufactured on the carrier film, they can be removed separately and folded in such a manner that the contact area in line with the bottom of the recesses is clear of the proximal extension of the electrode part.

### DETAILED DESCRIPTION OF THE INVENTION

The invention will now be explained in further detail in the description that follows of exemplary embodiments and with reference to the drawing, wherein:

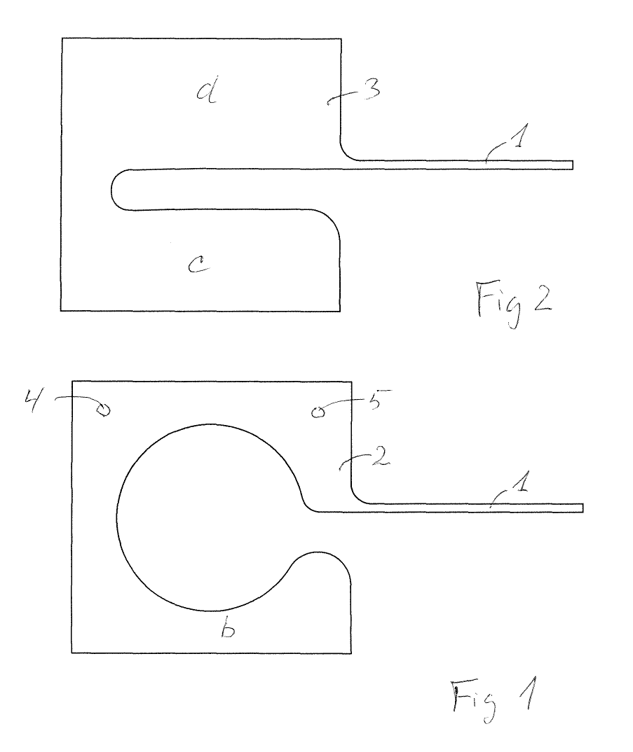

FIGS. 1 and 2 show two embodiments of the sensor according to the invention;

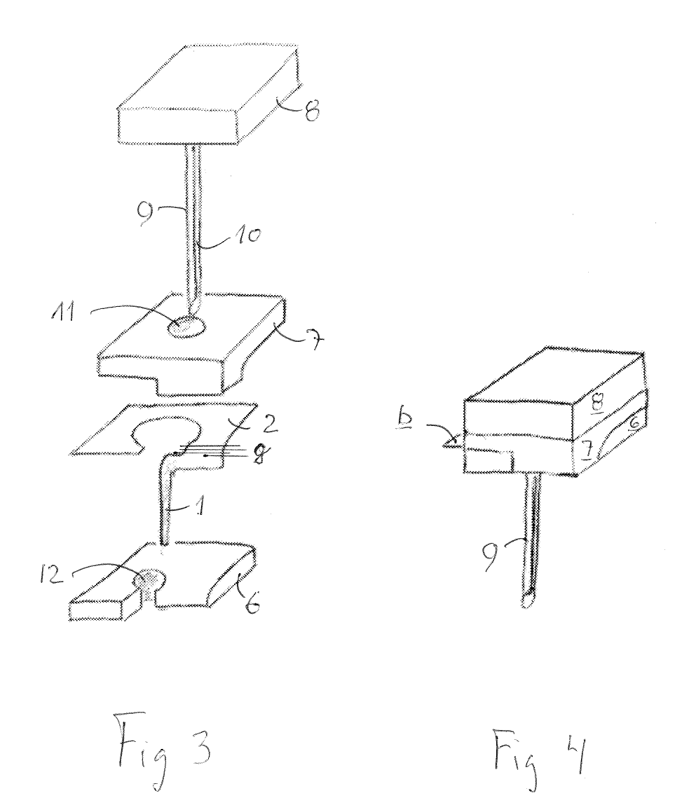

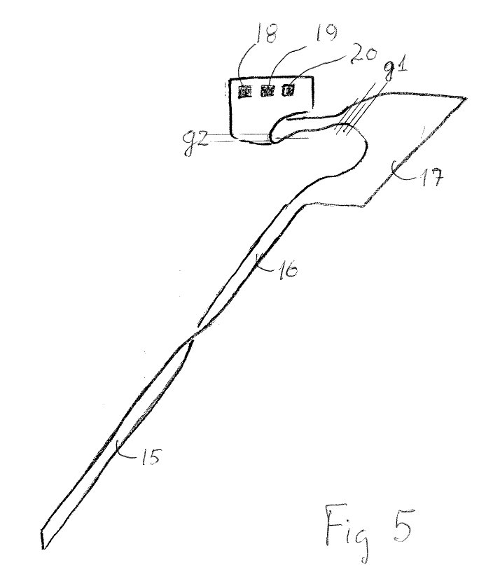

FIGS. 3 and 4 show a needle unit for insertion of eg the embodiments of the sensor shown in FIGS. 1 and 2, in exploded and assembled views, respectively;

FIG. 5 shows a further embodiment of the sensor according to the invention; while

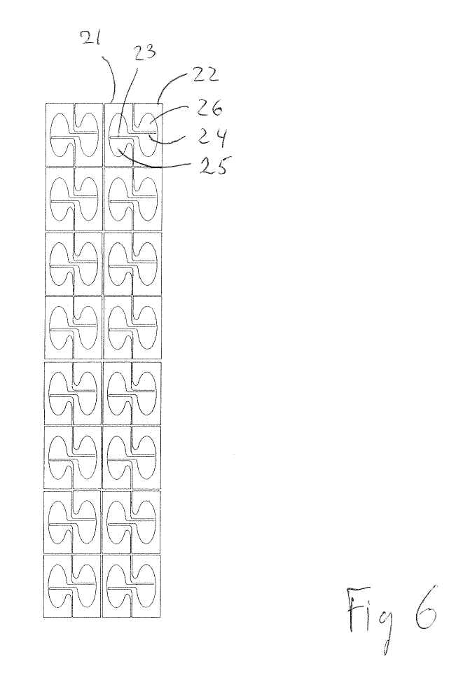

FIG. 6 serves to illustrate the method according to the invention.

The sensor shown in FIG. 1 comprises an electrode part 1 and a contact part designated by 2 in FIG. 1 and by 3 in FIG. 2.

The contact part 2 or 3 serves a number of purposes. Firstly, it serves to physically fixate the electrode part 1 and, next, the contact part 2 or 3 must be provided with electrical contact pads that are, in a manner known per se and as explained in the introductory part to the specification, electrically connected to the tip of the electrode part, where the electric electrodes are provided, cf the prior art cited in the introduction. Electrode pads can be arranged in various places on the electrode part; however, it is a major advantage of the invention that the area of the electrode part 2 or 3 is large enough to leave space for further applications, while simultaneously—as will be explained below in the context of the method according to the invention—the relatively expensive electrode material is used optimally.

Comparison of the embodiments shown in FIGS. 1 and 2 will reveal that the Ω-shaped embodiment shown in FIG. 1 is the most flexible one due to its comparatively larger recess compared to that of FIG. 2 which is rather rigid, but having, in return, an even larger contact part area than the embodiment shown in FIG. 1.

In accordance with the invention, it is a common feature of the embodiments shown in FIGS. 1 and 2 that the contact part is situated to both sides of an axial plane that contains a longitudinal axis of the elongate electrode part 1. For instance, this plane could be a plane at right angles to the paper plane in FIGS. 1 and 2, and it will readily be understood that, in this manner, it is possible to increase the area of the contact area, while simultaneously upholding the sensor as a compact unit. Thus, it will also be understood that the invention comprises any other wave-shaped or similar folding of the contact area, thereby providing such axial plane that the contact part is caused to be situated to both sides of that axial plane.

FIG. 1 shows a pair of apertures 4 and 5 that may serve as guide apertures for the sensor film. The electric contact pads will typically be situated in areas b or c in FIG. 1 or 2, respectively; however, they may also be present elsewhere on the contact part, eg in the area d of FIG. 2, where the area c could then be used for electric circuits which could be active circuits or passive circuits, such as a circuit layout that serves as an antenna to enable detection of information on the biochemical properties of the electrode. Owing to the relatively large area of the contact part, it is also an option—either by gluing or other technique—to laminate several other rigid or flexible material layers to the electrode part 2 and 3. Of course, subsequent folding of the laminated or non-laminated sensor film must be performed in such a manner as to allow the insertion needle of the electrode part 1 to be clear of the area of the electrode part which is situated in level with the bottom of the recess, as will appear more clearly from FIG. 3.

FIG. 3 shows an exploded view of a needle unit for cooperating with the sensor shown in FIG. 1. The unit comprises a bottom element 6, a middle element 7 and a top element 8. A needle 9 is secured to the top element 8 and features a slit 10 for receiving the electrode part 1 when the parts are assembled to the position shown in FIG. 4; said needle 9 extending through an aperture and an opening, 11 and 12, respectively, in the middle element and the bottom element, respectively. In practice the needle unit shown in FIG. 4 is used such that the needle is first inserted into the body until the bottom element 6 abuts on the skin, following which the top element 8 is pulled upwards, whereby the needle 9 is caused to follow along, while the electrode part 1 remains subcutaneously in the tissue.

It will be understood that if the sensor had the plane shape shown in FIG. 1, the needle would not be able to be clear of that part of the contact part which is located in level with the bottom of the recess, but this problem is obviated in that the contact part has been folded about a number of generatrices designated by g in FIG. 3. However, it will be noted that the variety is also encompassed by the invention that the needle travels quite closely past a point on the contact part, thereby enabling use of the needle for electrically short-circuiting the electrical conductors that connect the electrodes to the contact pads. Thereby the risk is almost eliminated of a subsequent sterilisation of the sensor destroying electronic circuits that were previously arranged on the sensor film. Therefore, it is highly perceivable that the bottom of the Ω-shaped contact part 2 had been folded back again and pressed elastically against the needle. This would enable sterilisation without vitiating the ability of the needle to be withdrawn.

FIG. 5 shows a further embodiment of the sensor according to the invention. This embodiment differs from the embodiments described so far on three points.

Firstly, it will appear that the electrode part is twisted to the effect that it comprises a distal end 15 and a proximal end 16, the latter being integral with a contact part 17. The twisting means that the electrode part is supported such in the slit 10 of the needle that the risk of tissue or hair dislocating the electrode from its position during the insertion is eliminated.

FIG. 5 also shows generatrices designated by g1 and shows that the contact part 17 is folded to the effect that the needle is able to avoid the contact part 17. In FIG. 5, these generatrices are essentially in parallel with the longitudinal axis of the electrode part, whereas the generatrices g in FIG. 3 were transversal to said longitudinal axis. In principle the electrode parts 2, 3 or 17 could be folded about generatrices having all possible orientations. FIG. 5 also shows generatrix-orientations g2 that show that the outermost end of the Ω-shaped contact part 17 is folded to the effect that the three contact pads 18, 19 and 20 are situated on a face part which is essentially perpendicular to the longitudinal axis of the electrode part. This may be particularly advantageous in the context of an embodiment which would be an alternative to the embodiment shown in FIG. 4, and wherein it is convenient to be able to push the contact pads down towards corresponding contact terminals in the same direction as the one in which the needle unit is inserted. As it is, this could not be accomplished by the embodiment shown in FIG. 4, where the contact part area b is shown to extend to the one side due to there being no support on the upwardly facing side of the film.

By folding the contact part 17 about the generatrix-lines g2, not only a convenient location of the contact pads is accomplished; it is also accomplished that one may advantageously arrange active electronic circuits on that part of the contact part 17 due to the P-N transitions of the circuits becoming essentially in parallel with the direction of irradiation, when the sensor shown in FIG. 5 is sterilised by means of electronic irradiation which will typically have directions perpendicular to the longitudinal axis of the electrode part.

FIG. 6 shows how the sensor films can be arranged on a sensor film substrate when sensors of the type shown in FIG. 1 are to be manufactured. Reference is made to the shown sensors 21 and 22, all the remaining sensors merely serving the purpose of illustrating the particular advantages obtained by the method according to the invention. Sensors 21 and 22 have an electrode part 23 and 24, respectively, and have recesses 25 and 26, respectively, and according to the invention and as will appear from FIG. 6, the sensors are situated such that the electrode part 24 from the sensor 21 extends into the recess 26 of sensor 22 and correspondingly for the other needle and recess.

In this manner it will be shown that the sensor film substrate can be used optimally with respect to utilization of the area. Of course, the larger the recesses 25, 26, the smaller the degree of utilization, whereby the highest possible and almost complete utilization of the sensor material can be obtained in accordance with the kind of sensor shown in FIG. 2; but, as mentioned above, there may be scenarios where a more flexible contact part is desired at the expense of the area of the contact part.

The further processing of the sensors will, of course, be to take the individual sensors off a support film, which may be an elongate band wound to a roller supply. Then the sensors can be folded to one of the many possible shapes, of which some were described above, following which the sensor film is arranged in combination with a needle unit.

1. A flexible sensor film comprising an elongate electrode part with a distal end for transcutaneous insertion into an organism by means of a needle and with a proximal end which is integral with a contact part of the sensor film, wherein the contact part extends to both sides of at least one axial plane that contains a longitudinal axis of the electrode part.

2. A sensor film according to claim 1, the contact part is essentially Ω-shaped.

3. A sensor film according to claim 1, wherein the film is folded such that the contact part is clear of the proximal extension of the electrode part.

4. A sensor film according to claim 1, wherein the contact part comprises a folding area having generatrices essentially in parallel with the electrode part.

5. A sensor film according to claim 1, wherein the contact part comprises a folding area having generatrices essentially transversally of the electrode part.

6. A sensor film according to claim 1 electric circuits are provided on the contact part.

7. A sensor film according to claim 1 contact pads are provided in an area of the contact part which is essentially perpendicular to the longitudinal axis of electrode part.

8. A sensor film according to claim 7, wherein the said area of the contact part is provided with active electronic components.

9. A flexible sensor film according to claim 1, wherein electric conductors are provided for coupling electrodes on the electrode part to contact areas on the contact part, characterised in that the conductors are exposed in line with the proximal extension of the electrode part, thereby enabling them to be short-circuited by the needle.

10. A flexible sensor film according to claim 1, wherein the sensor film is laminated and at least partially comprises two or more layers.

11. A flexible sensor film according to claim 2, wherein the contact area is Ω-shaped with filled external corners of which at least some have a guide opening for automatic mounting.

12. A flexible sensor film according to claim 1, wherein the electrode part of the sensor film is twisted somewhere between the distal and the proximal ends, thereby causing these ends to extend in essentially mutually perpendicular planes.

13. A method of manufacturing a flexible sensor film having a distal end and a proximal end that are coherent at a contact part of the sensor film, which method is wherein sensor elements are provided on a carrier film, wherein each sensor element comprises a contact part having a recess that extends into the contact part and comprises an electrode part that extends in a direction away from the contact part in line with the recess, and wherein the sensor elements are situated in pairs opposite each other, whereby an electrode part from the one element in the pair of elements extends into the recess in the second element in the pair of elements.

14. A method according to claim 13, wherein the area of the recess is comparatively large relative to the area of the contact part.

15. A method according to claim 13 wherein the area of the recess is comparatively small relative to the area of the contact part.

16. A method according to claim 13, wherein the carrier film is an elongate band.

17. A method according to claim 13 wherein the elements are removed from the film and folded such that the contact area in line with the bottom of the recess is clear of the proximal extension of the electrode part.
